(19) United States
(12) Patent Application Publication
(10) Pub. No.: US 2024/0245613 A1
(43) Pub. Date: Jul. 25, 2024

(54) PEPTIDE LIQUID DROPLETS AND METHODS OF USING THE SAME

(71) Applicant: RAMOT AT TEL AVIV UNIVERSITY LTD., Tel Aviv (IL)

(72) Inventors: Ayala LAMPEL, Tel Aviv (IL); Avigail-Elah BARUCH LESHEM, Tel Aviv (IL); Amit BEN-AMAR, Tel Aviv (IL)

(21) Appl. No.: 18/433,057

(22) Filed: Feb. 5, 2024

Related U.S. Application Data
(63) Continuation of application No. PCT/IL2022/050903, filed on Aug. 18, 2022.
(60) Provisional application No. 63/242,071, filed on Sep. 9, 2021.

(51) Int. Cl. A61K 9/50; A61K 47/42; C07K 7/06; C07K 7/08

(52) U.S. Cl. CPC A61K 9/5052; A61K 47/42; C07K 7/06; C07K 7/08

(57) ABSTRACT

The invention provides a peptide liquid droplet a composition the same and methods for the delivery of an active ingredient, as disclosed herein. The composition comprises a peptide liquid droplet and an active agent encapsulated in the peptide liquid droplet. Further provided are a method for encapsulation of an active agent in a peptide liquid droplet; method for manufacturing an active ingredient in the peptide liquid droplet and a method for the delive

[symbol]

### CROSS REFERENCE TO RELATED APPLICATIONS

This application is a Bypass Continuation of PCT Patent Application No. PCT/IL2022/050903 having International filing date of Aug. 18, 2022, which claims the benefit of priority of U.S. Provisional Patent Application No. 63/242,071, filed Sep. 9, 2021, the contents of which are all incorporated herein by reference in their entirety.

### REFERENCE TO AN ELECTRONIC SEQUENCE LISTING

The contents of the electronic sequence listing (sequence-listings-rmt-11.xml; Size: 25,158 bytes; and Date of Creation: Feb. 5, 2024) is herein incorporated by reference in its entirety.

### BACKGROUND OF THE INVENTION

The emerging field of liquid-liquid phase separation (LLPS) as the basis of biomolecular condensation formation inspired efforts to design materials that are formed by dynamic assemblies. Yet, the molecular level basis of protein LLPS is still not fully understood and typically studied by in vitro studies of complex intrinsically disordered proteins (IDPs) which are challenging to produce and typically require a number of expression-purification steps. Unlike proteins, simple changes to the chemical composition of peptides, even at the individual amino acid level, can significantly affect their assembly behaviour, allowing sequence-structure and structure-function relationships to be established.

Production of small molecule drugs, proteins and enzymes in the pharmaceutical industry mainly relies either on chemical synthesis or cell-based synthesis. Both strategies have critical limitations: chemical synthesis of small molecules results in low enantioselectivity, poor conversion rate, complicated reaction procedure and requires expensive metal catalysts. Cell-based production in bacteria, insects, or plant cells requires expensive and time-consuming purification steps.

There is a need, thus, for a cell-free technology for the production and encapsulation of various biomolecules using peptide-based liquid droplets. Further, there is a need for peptide droplets with customizable chemical microenvironments that enable controlled release and/or production of biomolecules.

### SUMMARY OF THE INVENTION

The invention provides LLPS-promoting peptides which form liquid droplets by simple or complex coacervation. By utilizing a minimalistic approach, the inventors of the invention developed several motives to promote intermolecular interactions. In some embodiments, there is provided a peptide liquid droplet and a composition comprising the same, wherein the peptide comprises two, three or more alternating arginine and glycine dyads (RGRGRG, SEQ ID No. 26) that provide the sequence charge and flexibility with one and one, two or more aromatic amino acids. In some embodiments, the aromatic amino acid is located at the end of the sequence. In some embodiments, the two or more aromatic amino acids are located at both ends of the arginine and glycine dyads to enhance π-π stacking interactions. The second motif is optional and includes the hydrophobic elastin-like peptide (ELP) repeating domain (WPGVG, SEQ ID No. 27). The presence of ELP enables to increase the mobility and dynamics of the droplets from 4.52E-14 (of WGR-1) to 2.36E-14 (of WGR-4). As can be seen, presence of ELP enhances the dynamics by about two-fold. The developed peptides were utilized to promote LLPS and were characterized by means of turbidity, optical microscopy, and confocal microscopy, where peptide diffusion and mobility were studied by fluorescence recovery after photobleaching (FRAP). Fluorescence measurement and microscopy were used for characterization of encapsulation of two fluorescent dyes and green fluorescent protein (GFP). The phase diagrams of the peptide's library emphasized the importance of the interactions between arginine and the aromatic amino acids to promote phase separation. Moreover, the results suggest that the strength of intermolecular interactions between arginine and aromatics directly affects the diffusion of the peptides inside the condense phase and the peptide polarity affects the encapsulation efficiency of the dyes to the condensed phase. The findings shed light on the chemical basis of peptide LLPS and provide insights into the sequence-structure relationship of peptide liquid droplets. The peptides of the invention open tremendous opportunities in biomedical applications by customizing the encapsulation efficiency of peptide liquid droplets based on their specific chemical properties.

In some embodiments, there is provided a peptide liquid droplet, wherein the peptide liquid droplet comprises a sequence comprising one, two, or more dyad of arginine-glycine (RG), one or more aromatic amino acid; and optionally an ELP domain.

In some embodiments, the one or more aromatic amino acid are located at each end of the one, two, or more dyad of arginine-glycine (RG).

In some embodiments, the aromatic amino acid is tyrosine, tryptophan, histidine or phenylalanine.

In some embodiments, the ELP domain is hydrophobic.

In some embodiments, the valine at the first position of the ELP domain is substituted by tryptophan so that WPGVG (SEQ ID No. 27) is formed. In some embodiments, V (valine) can be substituted with any non-polar amino acids i.e., alanine (A), leucine (L) or isoleucine (I) as well as with aromatic W, F, or Y.

In some embodiments, the peptide liquid droplet comprises between 2-10 dyads of RG.

In some embodiments, the peptide liquid droplet comprises 2, 3, 4, 5, 6, 7, 8, 9 or 10 dyads of RG.

In some embodiments, the peptide liquid droplet comprises

(SEQ ID No. 1)
WGRGRGRGWPGVGY;
(SEQ ID No. 3)
WGRGRGRGWPGVGF;
(SEQ ID No. 4)
WGRGRGRGWY;
(SEQ ID No. 5)
WGRGRGWPGVGY;
or
(SEQ ID No. 25)
WGRGRGRGWQGVGY;
(SEQ ID No. 6)
WGRGRGRWY.

In some embodiments, the peptide sequence is as set forth in SEQ ID Nos. 1, 3-23 and 25 as depicted in Table 1.

In some embodiments, the peptide liquid droplet encapsulates an active agent.

In some embodiments, there is provided a composition comprising a phase comprising the peptide liquid droplet as described herein and a dilute aqueous phase.

In some embodiments, there is provided a pharmaceutical composition comprising the peptide liquid droplet or the composition comprising thereof and a pharmaceutically acceptable carrier.

In some embodiments, the active agent is selected from the group consisting of proteins, (poly)peptides, carbohydrates, nucleic acids, lipids, chemical compounds, nanoparticles, and combinations thereof.

In some embodiments, the active agent is a pharmaceutical or diagnostic agent.

In some embodiments, the pH of the composition is between 5-10. In some embodiments, the pH is more than 5, 6, 7, 8, 9, or 10 or more.

In some embodiments, there is provided a method for the encapsulation of an active agent in a peptide liquid droplet, the method comprising: (a) mixing an aqueous solution of peptide liquid droplet, wherein the peptide liquid droplet as defined herein together with the active agent; and (b) inducing peptide liquid formation.

In some embodiments, the pH of the aqueous solution of the peptide liquid droplet is between 5-10. In some embodiments, the pH is more than 5, 6, 7, 8, 9, or 10 or more.

In some embodiments, the concentration of the peptide liquid droplet provided in the aqueous solution ranges between about 5 mM to 30 mM.

In some embodiments, there is provided a method for the delivery of an active agent, comprising: (i) providing a composition as described herein or a peptide liquid droplet as described herein and (ii) exposing the peptide liquid droplet to conditions that trigger the release of the active agent from therefrom.

In some embodiments, the conditions that trigger the release of the active agent are selected from the group consisting of elevated temperatures, pH changes or change to the ionic strength, guest molecules, enzymatic activation, exposure to release agents, exposure to light and combinations thereof.

In some embodiments, there is provided a method for treating disease in a subject in need thereof, comprising: (i) administering a composition comprising a peptide liquid droplet according to the embodiments of the invention; and (ii) exposing the peptide liquid droplet to conditions that trigger the release of the pharmaceutical or diagnostic agent.

In some embodiments, there is provided a complex peptide liquid droplet, wherein the peptide liquid droplet comprises: a sequence comprising one, two or more dyad of arginine-glycine (RG) and one or more aromatic amino acid. In some embodiments, an ELP domain mixed together with a negatively charged polymer is part of the complex.

In some embodiments, the negatively charged polymer is an RNA or DNA polymer, or any anionic polyelectrolyte.

In some embodiments, the RNA polymer is a poly U polymer or any anionic polyelectrolyte

In some embodiments, the complex peptide droplet comprises WGRGRGRWY (SEQ ID No. 6) or any of the peptides of SEQ ID. No. 1, SEQ ID. No. 3, SEQ ID. No. 4, SEQ ID. No. 5 or SEQ ID. No. 25 and a negatively charged polymer, such as anionic polyelectrolyte, RNA or DNA. In some embodiments, the complex peptide droplet comprises

(SEQ ID No. 1)
WGRGRGRGWPGVGY;
(SEQ ID No. 3)
WGRGRGRGWPGVGF;
(SEQ ID No. 4)
WGRGRGRGWY;
(SEQ ID No. 5)
WGRGRGWPGVGY;
or
(SEQ ID No. 25)
WGRGRGRGWQGVGY;
(SEQ ID No. 6)
WGRGRGRWY.

and poly U polymer.

In some embodiments, the complex peptide droplet comprises WGRGRGRWY (SEQ ID No. 6) and poly U polymer. In some embodiments, the complex peptide droplet comprises WGRGRGRWY (SEQ ID No. 6) or any of the peptides of SEQ ID. No. 1, SEQ ID. No. 3, SEQ ID. No. 4, SEQ ID. No. 5 or SEQ ID. No. 25 and RNA or DNA polymer or any anionic polyelectrolyte.

In some embodiments, the complex peptide droplet of the invention is mixed with tyrosinase. In some embodiments, a melanin-like material is formed from oxidation and polymerization of the tyrosine within the peptide WGRGRGRWY or any one of the peptide having amino acids as set forth in any one of SEQ ID Nos: 1, 4, 5, and 6, any one of the peptides having sequences as set forth in SEQ ID Nos: 7, 8, 10, 11, 12, 16, 17. 18. 19, 20, 22, 23, and 25 or the peptide liquid droplet, wherein the peptide comprises two, three, or more alternating arginine and glycine dyads that provide the sequence charge and flexibility with one and one, two or more aromatic amino acids provided that the peptide contains at least one tyrosine.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1: peptide liquid droplets. The top panel of FIG. 1 presents the chemical structure of WGR-1. Color code: aromatic side chain in blue, basic side chains of arginine in turquoise, and the ELP domain in orange. The middle panel is a schematic representation of the designed system: WGR-1 forms liquid droplets through liquid-liquid phase separation by simple coacervation triggered by salt or specific pH, where the formed droplets can partition various biomolecules. The bottom panel shows possible intermolecular interactions of WGR-1 including (from left to right) π-π stacking between Trp and Trp-Tyr side chains, and between Trp and Tyr side chains, and π-π interactions between Arg and Trp or Arg and Tyr side chains.

FIGS. 2A and 2B: Peptide sequence controls liquid-liquid phase separation propensity. FIG. 2A shows a table of the designed peptide sequences. FIG. 2b top panel demonstrates a phase diagram of the peptides from left to right: WGR-1, WGR-2, WGR-3, WGR-4, WGR-5 and WGK as a function of peptide concentration and pH, in the presence of 0.2 M NaCl. LLPS was not observed for WGR-2 and WGK. FIG. 2b bottom panel shows confocal microscopy bright field images of peptide liquid droplets formed at a concentration of 20 mM in Tris buffer at pH 8 with 0.2 M NaCl. Scale bar=20 μm. Inset: macroscopic images of the peptide solutions.

FIGS. 3A, 3B and 3C: The dynamic behavior of peptide droplets is affected by the strength of intermolecular interactions. Fluorescence recovery after photo-bleaching (FRAP) analysis of WGR-1, WGR-3, WGR-4, and WGR-5 showing the FRAP recovery of the different peptides by (FIG. 3a) confocal microscopy images; (FIG. 3b) fluorescence intensity over time; (FIG. 3c) and diffusion coefficient.

FIGS. 4A, 4B: Partitioning of fluorescent biomolecules within droplets depends on peptide polarity. Confocal microscopy images (FIG. 4a) of fluorescein, Rhodamine B and GFP partitioning within peptide droplets. Scale bar=10 μm. Inset: macroscopic images of WGR-1 samples with fluorescent payloads before (left) and after (right) centrifugation. Encapsulation efficiency (EE) analysis of (FIG. 4b) fluorescein, Rhodamine B and GFP, calculated from absorbance measurements of bulk solutions. Values represent an average of three different measurements, error bars are indicated.

FIG. 5 demonstrates a phase diagram of the LLPS propensity of WGR-6-poly-U at varying peptide and RNA concentrations. Values represent the turbidity (OD) of the samples.

FIGS. 6A, 6B and 6C, 6D are optical microscopy images of droplets formed by complex coacervation of WGR-6 (2 mM) with 0.25 mg/ml (FIG. 6a), 0.5 mg/ml, (FIG. 6b), 1 mg/ml (FIG. 6c), and 2 mg/ml (FIG. 6d) of poly-U. Scale bars=20 μm.

FIGS. 7A and 7B demonstrate that peptide:RNA droplets sequester tyrosinase. Confocal microscopy images (FIG. 7a) and intensity quantification (FIG. 7b) of Atto633-labeld tyrosinase partitioning in WGR-6:poly-U droplets (2 mM, 1 mg/ml), measured at λex=640 nm. Values in (FIG. 7b) represent average fluorescence intensity of 10 droplets or spots at the surrounding phase.

FIGS. 8A and 8B show UV-Vis absorbance analysis of peptide:poly-U (2 mM, 1 mg/ml) droplets over oxidation time (FIG. 8a) and macroscopic images of the materials over oxidation time (FIG. 8b).

FIGS. 9A, 9B demonstrate emerging fluorescence of oxidized peptide-RNA droplets. Confocal microscopy images (FIG. 9a) of the intrinsic fluorescence of droplets formed by WGR-6 (2 mM) and poly-U (1 mg/ml) following 1 h, 5 h, 24 h, 48 h, and 72 h of oxidation. Scale bars=5 μm. FIG. 9b is a quantitative analysis of the fluorescence intensity of droplets or their surrounding phase at 1 h, 5 h, 24 h, 48 h, and 72 h of oxidation measured at λex=405 nm or λex=640 nm. Values are average of 10 droplets spots at the surrounding phase.

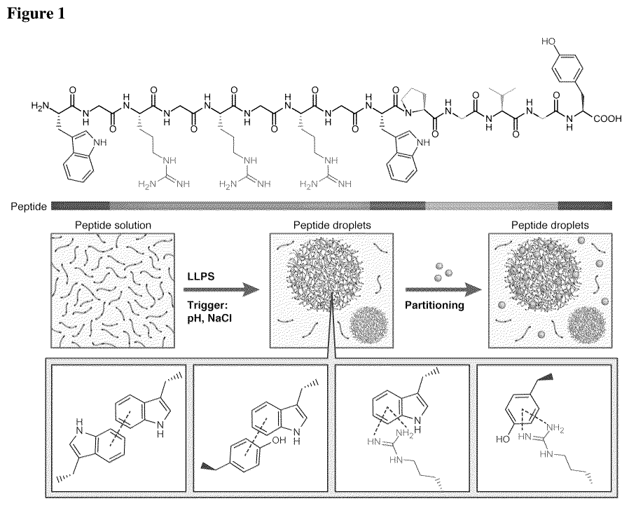

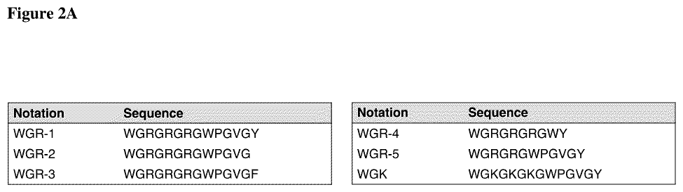

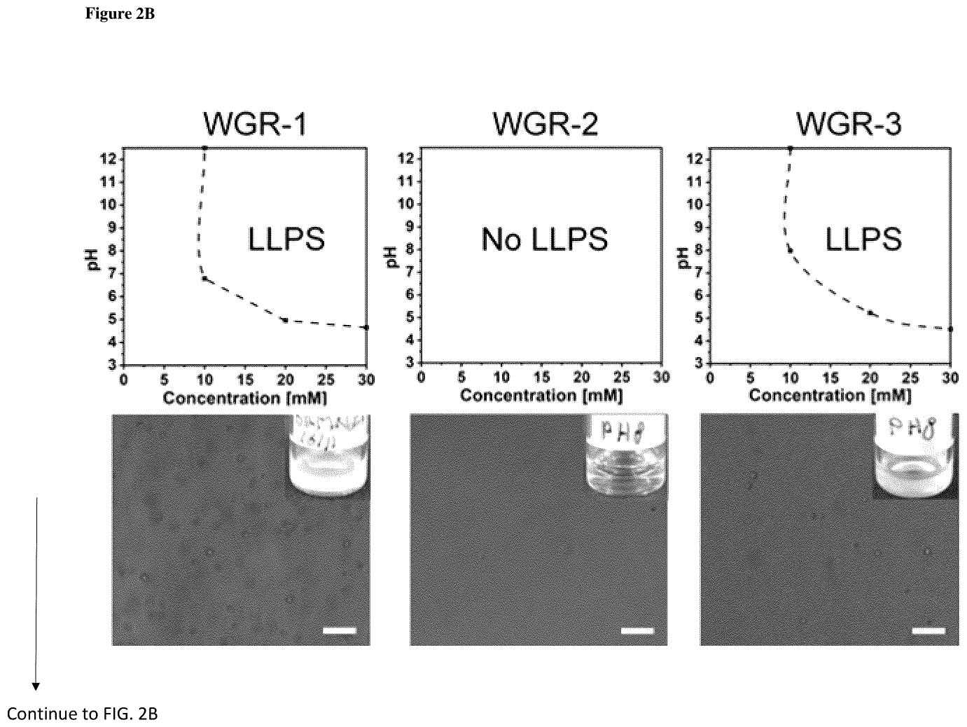

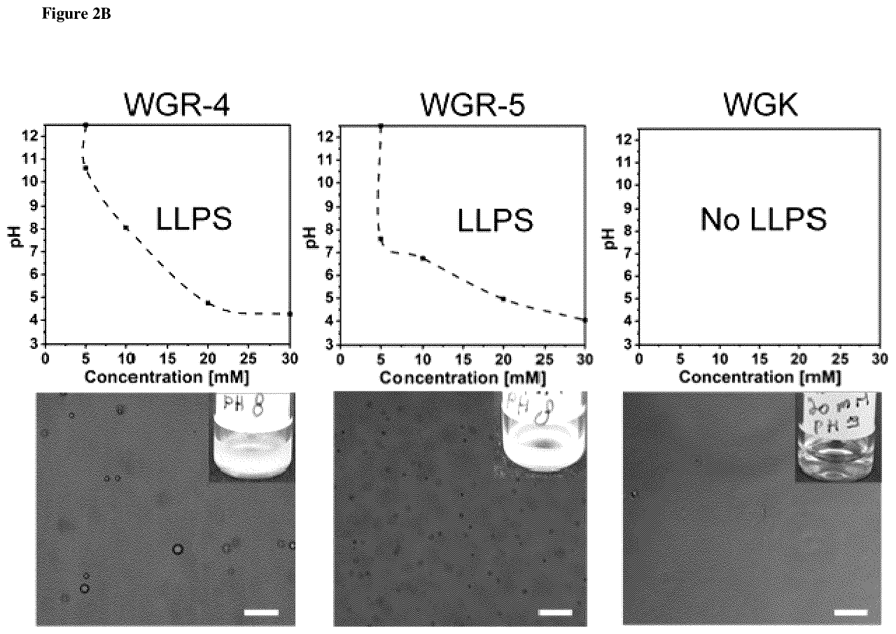

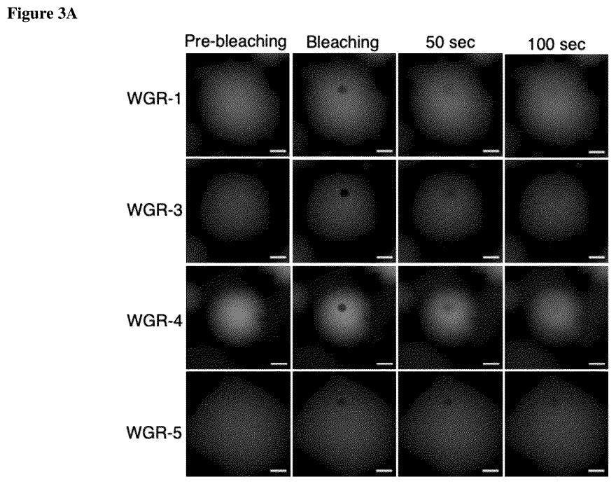

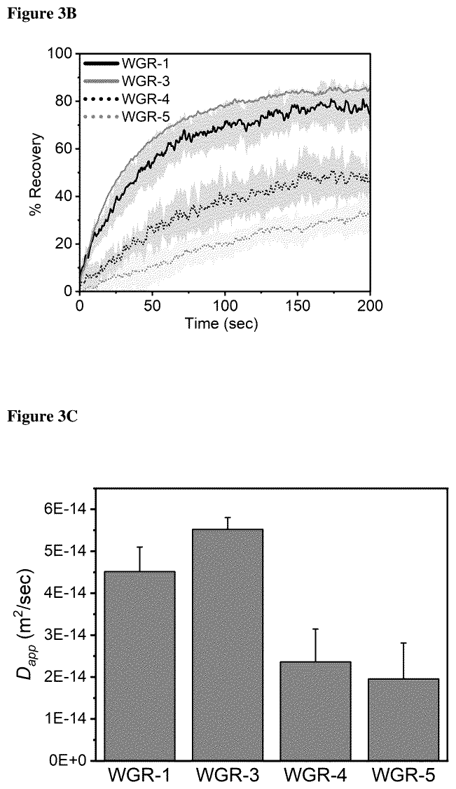

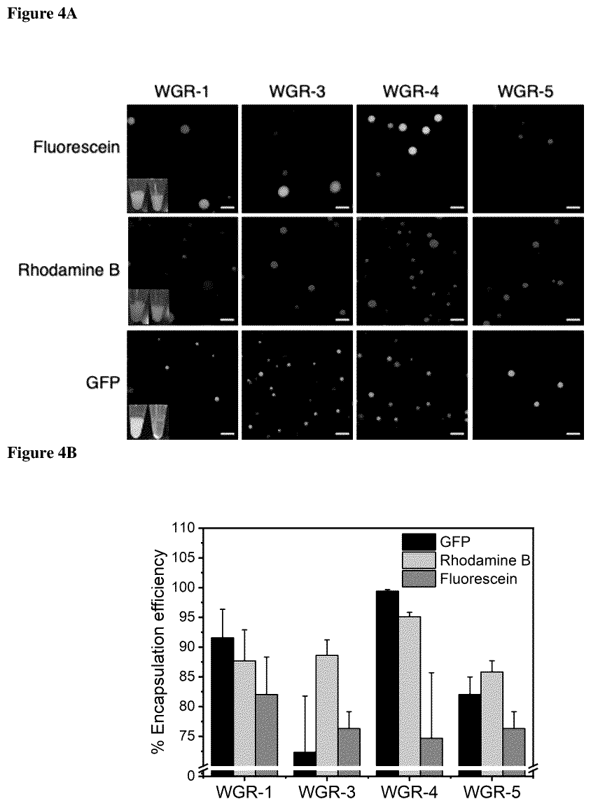

### DETAILED DESCRIPTION OF THE EMBODIMENTS

In some embodiments, there is provided a library of LLPS-promoting peptides which form liquid droplets by simple coacervation. By utilizing a minimalistic approach, we incorporated several motives to promote intermolecular interactions, including alternating arginine-glycine dyads and aromatic amino acids to promote π-π stacking interactions. The peptides' LLPS behavior was characterized by turbidity measurements, optical and confocal microscopy. Peptide diffusion and mobility were studied by fluorescence recovery after photobleaching (FRAP) analysis. These analyses show that the peptide sequence controls LLPS propensity where the interactions between the side chain of arginine and that of the aromatic amino acids are critical for LLPS. Moreover, the results suggest that the strength of arginine-aromatics interactions directly affect the diffusion rate of the peptides inside the condense phase. Encapsulation efficiency analysis showed that peptide polarity controls the partitioning of fluorescent dyes and green fluorescent protein (GFP) within droplets. The surprising findings shed light on the chemical basis of peptide LLPS and provide insights into the sequence-structure relationship of peptide liquid droplets. Furthermore, the resulted peptide liquid droplets open tremendous opportunities in biomedical applications by customizing the encapsulation efficiency of peptide droplets based on their specific chemical properties.

In some embodiments, there is provided a peptide lipid droplet, wherein the peptide liquid droplet comprises: a sequence comprising two or more dyad of arginine-glycine (RG), aromatic amino acids to promote pi-pi interactions with the arginine, and optionally an ELP domain.

In some embodiments, the aromatic amino acid is one on more of tyrosine, histidine tryptophan or phenylalanine and any combination thereof.

As can be seen from the Examples, the terminal tyrosine is critical for LLPS and droplet formation, as omitting this amino acid completely arrested LLPS. Substituting the tyrosine with phenylalanine recovered LLPS but shifted the phase diagram boundaries so the critical pH for LLPS is slightly higher. In addition, arginine is critical for LLPS, as substituting arginine with lysine completely inhibited LLPS and droplet formation. Thus, in some embodiments, the peptide liquid droplet comprises: a sequence comprising two or more dyad of arginine-glycine (RG), aromatic amino acids to promote pi-pi interactions with the arginine, and optionally an ELP domain, wherein the peptide sequence is ended with tyrosine.

In some embodiments, the ELP domain is WPGVG (SEQ ID No. 27) or WQGVG (SEQ ID No. 28. In some embodiments, V may be substituted by non-polar amino acid.

In some embodiments, the one or more aromatic amino acid may be located at each end and may be similar or different.

In some embodiments, the peptide liquid droplet comprises between 1, 2, 3, 4, 5, 6, 7, 8, 9, or 10 dyads of RG.

In some embodiments, the peptide liquid droplet comprises

(SEQ ID No. 1)
WGRGRGRGWPGVGY;
(SEQ ID No. 3)
WGRGRGRGWPGVGF;
(SEQ ID No. 4)
WGRGRGRGWY;
(SEQ ID No. 5)
WGRGRGWPGVGY;
or
(SEQ ID No. 25)
WGRGRGRGWQGVGY;
(SEQ ID No. 6)
WGRGRGRWY.

In some embodiments, the peptide comprises between 5-10 amino acids. In some embodiments, the peptide comprises 5, 6, 7, 8, 9 or 10 amino acids. In some embodiments, the peptide comprises between 10-20 amino acids. In some embodiments, the peptide comprises 10, 11, 12, 13, 14, 15, 16, 17, 18, 19 or 20 amino acids. In some embodiments, the peptide comprises between 20-50 amino acids. In some embodiments, the peptide comprises between 5-50 amino acids. In some embodiments, the peptide comprises between 50-100 amino acids. In some embodiments, the peptide comprises between 100-300 amino acids.

In some embodiments, the sequence of the peptide liquid droplet is as set forth in Table 1.

TABLE 1

| No. | Peptide | SEQ ID. No. |
|---|---|---|
| 1 | WGRGRGRGWPGVGY | SEQ ID No. 1 |
| 2 | WGRGRGRGWPGVGF | SEQ ID No. 3 |
| 3 | WGRGRGRGWY | SEQ ID No. 4 |
| 4 | WGRGRGWPGVGY | SEQ ID No. 5 |
| 5 | WGRGRGRWY | SEQ ID No. 6 |
| 6 | WGRGRGRGY | SEQ ID No. 7 |
| 7 | WGRGRGWY | SEQ ID No. 8 |
| 8 | WGRGRGRGWPGVGW | SEQ ID No. 9 |
| 9 | WGRGRGRGYPGVGY | SEQ ID No. 10 |
| 10 | YGRGRGRGYPGVGY | SEQ ID No. 11 |
| 11 | WGRGRGRGFPGVGY | SEQ ID No. 12 |
| 12 | WGRGRGRGFPGVGF | SEQ ID No. 13 |
| 13 | FGRGRGRGFPGVGF | SEQ ID No. 14 |
| 14 | WGRGRGRGWF | SEQ ID No. 15 |
| 15 | FGRGRGRGWY | SEQ ID No. 16 |
| 16 | WGRGRGRGYY | SEQ ID No. 17 |
| 17 | FGRGRGRGFY | SEQ ID No. 18 |
| 18 | WRGRGRGWY | SEQ ID No. 19 |
| 19 | WRGRGWY | SEQ ID No. 20 |
| 20 | WRGRGWW | SEQ ID No. 21 |
| 21 | WRGRGFY | SEQ ID No. 22 |
| 22 | FRGRGFY | SEQ ID No. 23 |
| 24 | WGRGRGRGWQGVGY | SEQ ID No. 25 |

In some embodiments, the peptide liquid droplet encapsulates an active agent.

In some embodiments, there is provided a composition comprising a phase comprising the peptide liquid droplet as described herein and a dilute aqueous phase. In some embodiments, there is provided a pharmaceutical composition comprises the peptide liquid droplet of the invention, wherein the peptide liquid droplet encapsulates an active agent; and a pharmaceutically acceptable carrier.

In some embodiments, the active agent is selected from the group consisting of proteins, (poly)peptides, carbohydrates, nucleic acids, lipids, chemical compounds, nanoparticles, and combinations thereof.

In some embodiments, the active agent is a pharmaceutical or diagnostic agent.

In some embodiments, the pH of the composition is between 5-10. In some embodiments, the pH of the composition is more than 5, 6, 7, 8, 9 or 10.

In some embodiments, the pH of the aqueous solution of the peptide liquid droplet is between 6-9.

In some embodiments, the pH of the aqueous solution of the peptide liquid droplet is between 7-9.

In some embodiments, there is provided a method for the encapsulation of an active agent in a peptide liquid droplet, the method comprising: (a) mixing an aqueous solution of peptide liquid droplet, wherein the peptide liquid droplet as defined herein together with the active agent; and (b) inducing peptide liquid formation. The active agent can either be added to existing droplets and partition within them or, added to the peptide solution prior to droplet formation and partition to the droplets upon their formation.

In some embodiments, the pH of the aqueous solution of the peptide liquid droplet is between 5-10.

In some embodiments, the pH of the aqueous solution of the peptide liquid droplet is between 6-9.

In some embodiments, the pH of the aqueous solution of the peptide liquid droplet is between 7-9.

In some embodiments, the concentration of the peptide liquid droplet provided in the aqueous solution ranges between about 5 mM to 30 mM. In some embodiments, the concentration of the peptide liquid droplet provided in the aqueous solution ranges between about 5 mM to 10 mM. some embodiments, the concentration of the peptide liquid droplet provided in the aqueous solution ranges between about 10 mM to 20 mM. some embodiments, the concentration of the peptide liquid droplet provided in the aqueous solution ranges between about 20 mM to 30 mM.

In some embodiments, there is provided a complex peptide liquid droplet, wherein the peptide liquid droplet comprises: a sequence comprising one, two or more dyad of arginine-glycine (RG), one or more aromatic amino acid; and optionally an ELP domain mixed together with a negatively charged polymer.

In some embodiments, the negatively charged polymer is an RNA or DNA polymer.

In some embodiments, the RNA polymer is a poly U polymer.

In some embodiments, the complex peptide droplet comprises

(SEQ ID No. 1)
WGRGRGRGWPGVGY;
(SEQ ID No. 3)
WGRGRGRGWPGVGF;
(SEQ ID No. 4)
WGRGRGRGWY;
(SEQ ID No. 5)
WGRGRGWPGVGY;
or
(SEQ ID No. 25)
WGRGRGRGWQGVGY;
(SEQ ID No. 6)
WGRGRGRWY

and poly U polymer.

In some embodiments, the complex peptide droplet comprises WGRGRGWPGVGY (SEQ ID No. 5) and poly U polymer. In some embodiments, the complex peptide droplet comprises any one of the amino acids in SEQ ID No. 1, 2, 3, 4, 5, or 6 and poly U polymer.

In some embodiments, the complex peptide droplet comprises any one of the amino acids in SEQ ID No. 1, 2, 3, 4, 5, or 6 or the amino acid sequences as provided in SEQ ID Nos. 78, 9, 10, 11, 12, 13, 14, 15, 16, 17, 18, 19, 20, 21, 22, 23, and 25 or a peptide comprises two, three or more alternating arginine and glycine dyads that provide the sequence charge and flexibility with one and one, two or more aromatic amino acids and poly RNA or DNA or any other negatively charged polymer.

In some embodiments, the complex peptide droplet of the invention is mixed with tyrosinase.

In some embodiments, a melanin-like material is formed from oxidation and polymerization of the tyrosine within the peptide WGRGRGWPGVGY (SEQ ID NO. 5), or with any one of the amino acids in SEQ ID No. 1, 4, 5, or 6

In some embodiments, there is provided a method for the delivery of an active agent, comprising: (i) providing a composition or a peptide liquid droplet as described herein; and (ii) exposing the peptide liquid droplet to conditions that trigger the release of the active agent from therefrom.

In some embodiments, the conditions that trigger the release of the active agent are selected from the group consisting of elevated temperatures, pH changes, ionic strength, guest molecule, exposure to light (UV irradiation), exposure to release agents, and combinations thereof.

In some embodiments, there is provided a method for treating disease in a subject in need thereof, comprising: (i) administering a composition comprising a peptide liquid droplet as described herein; and (ii) exposing the peptide liquid droplet to conditions that trigger the release of the pharmaceutical or diagnostic agent.

In some embodiments, the conditions that trigger the release of the pharmaceutical or diagnostic agent are selected from the group consisting of elevated temperatures, pH changes, exposure to release agents, and combinations thereof.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### Experimental Procedures

### Materials

Peptides were custom synthesized, then purified by high performance liquid chromatography to 95% and supplied as lyophilized powders by Genscript, Hong Kong. Unless otherwise specified, all reagents were purchased from Sigma-Aldrich (Rehovot, Israel) and were of the highest available purity GFP (made by abcam) was purchased from Zotal as a solution of 1 mg/ml in 0.316% Tris HCl, 10% Glycerol at pH 8. Aliquots of 7.5 μL were separated and stored at −20° C. until use.
Fluorescein—Holland Moran
Rhodamin B—Holland Moran
NaCl—BioLab
NaOH—BioLab
HCl—BioLab
Tris—Sigma
Ammonium bicarbonate—Holland Moran
Citrate—tzamal
Citric acid—tzamal

### Phase Diagrams and Peptide Liquid Droplets Preparation

150 μl of 5, 10, 20 and 30 mM peptide solutions were prepared in 20 mM of the suitable buffer (citrate buffer for a pH range of 3-6, tris buffer for pH range of 7-9 and ammonium bicarbonate to a pH range of 9-12) with 0.2 M NaCl. The pH was increased gradually until a turbidity appeared and measured as described below. The turbidity of 35 μl was measured in triplicates at λ=500 nm. This process repeated at higher pH, the pH increased until the turbidity faded, and a sediment appeared. All experiments were performed in triplicates and the presented value is the average.

### Turbidity Measurements

150 μl of 20 μM peptide solutions were prepared in 20 mM Tris buffer. The pH was adjusted to the desired value of 6, 7, 8, 9, 10 and 11. The turbidity of 35 μl was measured in triplicates at λ=500 nm.

### Imaging

All samples were imaged in a 96 Well Black Glass bottom plate, glass 1.5H (made by Hangzhou Xinyou, and purchased from Danyel Biotech) 10 minutes after preparation. The images were taken by Zeiss Zen 900 confocal microscope with ×20/0.8 NA Plan-Apochromat air objective. The light microscopy images were taken at PMT mode. PMT imaging were taken with 561 nm laser and fluorescence imaging were taken with 488, 561 and 405 nm lasers for fluorescein, rhodamine B and GFP, respectively.

Fluorescence Recovery after Photobleaching (FRAP)

FRAP experiments were performed by a Zeiss Zen 900 confocal microscope with ×20/0.8 NA Plan-Apochromat air objective. A circular area with radius of 2.5 μm was bleached with a 488 nm laser 100% intensity at 10 iterations; subsequent recovery of the bleached area was recorded with a 488 nm laser. Photobleaching correction and recovery time were calculated using OriginLab. The final FRAP recovery curve is the average of recovery curves collected from five separate droplets.

### Encapsulation Efficiency

1 mM Stock solutions of the fluorescein and Rhodamine B dye molecules were prepared in 20 mM Tris buffer. A 20 mM peptide coacervated solutions were prepared in 20 mM Tris+0.2 M NaCl at pH 8. A 148.5 μl of peptide solution was mixed with 1.5 μl of dye solution in a 1.5 ml Eppendorf tube and was pipetted. The 15 μl of GFP solution was added to 135 μl of peptide solution and was mixed in a 1.5 ml Eppendorf tube and was pipetted. After 10 minutes the samples were centrifuged at 15,000 rcf for 10 minutes. A volume of 120 μl from the supernatant was collected and vortexed and then the absorbance of 35 μl triplicates was measured in a 384 well black plate by Biotek H1 synergy plate reader (purchased from Lumitron, Israel). For GFP, a 7.5 μl of 20 mM Tris buffer to a 7.5 μl GFP aliquot from the purchased stock solution. The 15 μl of GFP solution was added to 135 μl of peptide solution and was mixed in a 1.5 ml Eppendorf tube and was pipetted. The concentration of GFP at the supernatant was measured via fluorescence. All experiments were performed in triplicate. The concentration of the supernatant solutions determinate by calibration curves. Imaging was made to 30 μl of uncentrifuged samples.

$$\% EE = \frac{{C}_{T} - {C}_{sup}}{{C}_{T}}$$

### Example 1

### Peptide Design and Coacervate Characterization

A primary peptide sequence that is combined of two motifs considering that the characteristics that affect most of the phase separation through cation-π interactions are: 1) aromatic/aliphatic amino acids ratio 2) molecular weight 3) arginine content, were designed. Since a low molecular weight peptide was aimed to, it was necessary to increase the arginine and aromatic content as much as possible in a short peptide sequence. Hence, the first motif is alternating arginine and glycine (GRGRGRG), provided the sequence charge and flexibility and two more aromatic amino acids were corporate at both ends of the sequence to enhance π-π stacking interactions. The second motif is the hydrophobic ELP domain (WPGVG). The received sequence was WGRGRGRGWPGVGY and is donated as WGR-1.

A phase diagram was created by increasing the pH at various peptide concentrations until a visible turbidity occurred. The turbidity was measured as the absorbance at λ=500 nm. To confirm that the turbidity occurred as a result of coacervation rather than precipitation, we performed an optical microscopy analysis of 20 mM peptide solution at pH 8. Increasing peptide concentrations decreased the pH which triggered LLPS, as shown by visible turbidity and microdroplets formation, while no phase separation was observed at concentration lower than 10 mM.

Five more peptides were designed. The peptides sequences are presented in Table 1. Removing the tyrosine (Y) from the peptide WGR-1 (denoted WGR-2) arrested phase separation. It is reasonable to assume that the π-π stacking interactions are taking a major part in the process of LLPS, hence, reducing one aromatic amino acid decreased dramatically the peptide's self-assembly propensity. At WGR-3, the tyrosine was exchange to phenylalanine to determine if the phenol side chain is superior to benzyl and to obtain a more inherent sequence for future applications. Hence, it was reasonable that the peptide WGR-3, where the tyrosine was replaced in phenylalanine, created LLPS at higher pH value comparing to WGR-1. Correspondingly, WGK, which holds three lysines instead the three arginines, showed no visual turbidity and the microscope images revealed only some aggregates. WGR-4 sequence includes a truncation of the ELP motif (excluded to the tryptophan residue to remain the number of aromatic amino acids). Surprisingly, WGR-4 although obtaining a truncation of the sequence, exhibit phase separation at more moderate conditions. The sequence WGR-5 which includes a truncation of one RG repetition inform us on the importance of the number charge residue and net charge perform a phase separation at similar conditions to WGR-4. Both of the later peptides show phase separation at concentration low as 5 mM and at lower pH values at higher peptide concentration.

| Notation | Sequence | SEQ ID NO. |
|---|---|---|
| WGR-1 | WGRGRGRGWPGVGY | SEQ ID No. 1 |
| WGR-2 | WGRGRGRGWPGVG | SEQ ID No. 2 |
| WGR-3 | WGRGRGRGWPGVGF | SEQ ID No. 3 |
| WGR-4 | WGRGRGRGWY | SEQ ID No. 4 |
| WGR-5 | WGRGRGWPGVGY | SEQ ID No. 5 |
| WGK | WGKGKGKGWPGVGY | SEQ ID No. 24 |

### Example 2

### Mechanical Characterization Through FRAP

A fluorescence resonance after photo bleaching (FRAP) used to evaluate the diffusion of the droplets. The droplets were prepared with 0.5% of FITC labeled peptide and were observed by confocal microscope. The apparent diffusion coefficients were calculated as followed:

$${D}_{app} = \frac{{r}^{2}}{t}$$

Where t is recovery time. The calculated apparent diffusion coefficients of the peptides were at the range of 0.93*10⁻¹⁴-5.52*10⁻¹⁴ m² sec⁻¹ as presented in Table 3. Out of the four LLPS-promoting peptides, WGR-3 (substitution of Tyr with Phe) has the largest apparent diffusion coefficient (D), more than 5-fold larger than that of WGR-5 and slightly larger than that of WGR-1. The higher D of WGR-3 compared to WGR-1 correlates with the LLPS propensity of the two peptides, suggesting that the higher mobility of WGR-3 is a result of weaker interactions between Phe and Arg compared to those of Tyr and Arg. WGR-4 has a lower diffusion coefficient than WGR-1, suggesting that the ELP domain interferes sterically with the interactions between the aromatic amino acid side chains, or between the aromatics and Arg, and thus, removing this domain might increase the accessibility of the aromatics and Arg groups. The lowest diffusion of WGR-5 indicates that decreasing the electrostatic repulsion by reducing the net charge of the peptide from +3 to +2 increases the strength of intermolecular interactions between the peptide building blocks and as a result, significantly lowers the mobility and dynamics of the droplets. Moreover, these results suggest that in the absence of the aliphatic ELP domain, cation-π or π-π interactions between Arg and the aromatic side chain are the dominant driving force for droplet formation. As these interactions are short-range, they can result in higher friction between the peptides molecules, and in turn, reduced peptide diffusion and droplet dynamics.

TABLE 3
Apparent diffusion coefficient

| | WGR-1 | WGR-3 | WGR-4 | WGR-5 |
|---|---|---|---|---|
| D | 4.51787E−14 | 5.52E−14 | 2.36E−14 | 9.27E−15 |
| error | 5.82491E−15 | 2.84E−15 | 7.85E−15 | 6.07E−15 |

### Example 3

### Encapsulation of Fluorescent Biomolecules

LLPS are widely known for the ability to encapsulate small molecules or proteins. This characteristic is important for drug delivery and to exploits coacervate as microreactors. In order to investigate the encapsulation capacity of the designed peptides two fluorescent dyes were used (fluorescein and rhodamine B) and the protein GFP. The peptides WGR-1, WGR-3, WGR-4 and WGR-5 were chosen for this experiment for their diverse chemical characteristics. The encapsulation efficiency was measured via bulk measurements and images of the droplets with the dyes were taken in a fluorescence confocal microscope.

The encapsulation efficiency of the dyes was ranged at 72-98% which indicates of higher dyes concentration inside the droplets comparing to outside. This stands with the fluorescence confocal microscope images that showed a higher intensity of the fluorescence dyes inside the droplets.

Among the coacervated peptides, WGR-3, which hold Phe residue instead of Tyr, was shown to be the less favorite for inducing phase separation. Hence, it is with reason that it has the lowest encapsulation efficiency of fluorescein and of GFP with a less favorite intermolecular interactions. Moreover, there was no significant difference between WGR-1 and WGR-4 encapsulation efficiency suggesting that the truncation of the aliphatic residues does not affect the interaction with the aromatic molecule, fluorescein. However, for the hydrophilic protein GFP, the hydrophilic peptide, WGR-4, demonstrated the highest encapsulation efficiency. WGR-5 obtained a compromised encapsulation efficiency, assumingly, since it is lack of one Arg-Gly dyad there are less interactions with the hydrophilic surface of the protein.

Rhodamine B is similar to fluorescein by structure; however, it has one positive charge which can participate at the cation-π interactions with the peptides and by thus produce a greater encapsulation

efficiency comparing to fluorescein. Since all of the measured peptides obtain the same number of aromatic residues, the encapsulation efficiency of Rhodamine B is similar for all four peptides.

### Example 4

### Melanin-Like Material Formed by Peptide Biomolecular Condensate

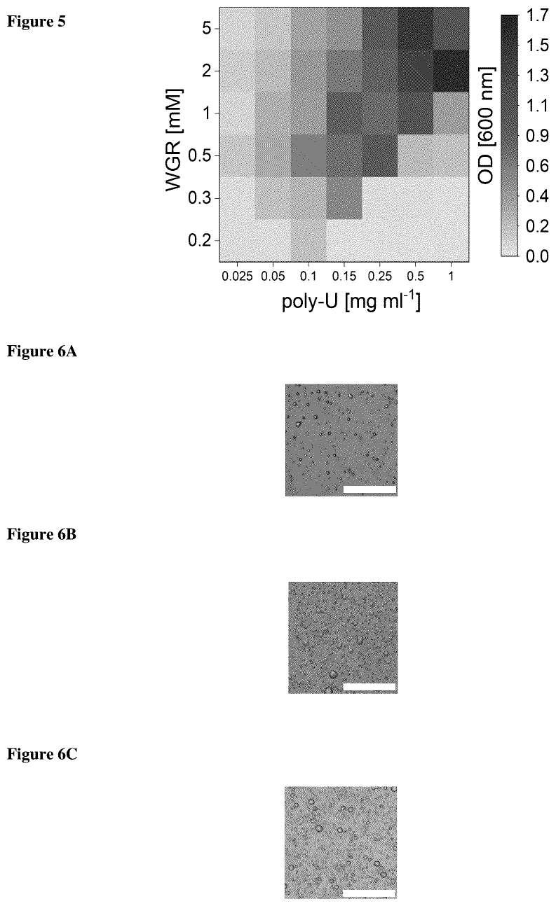

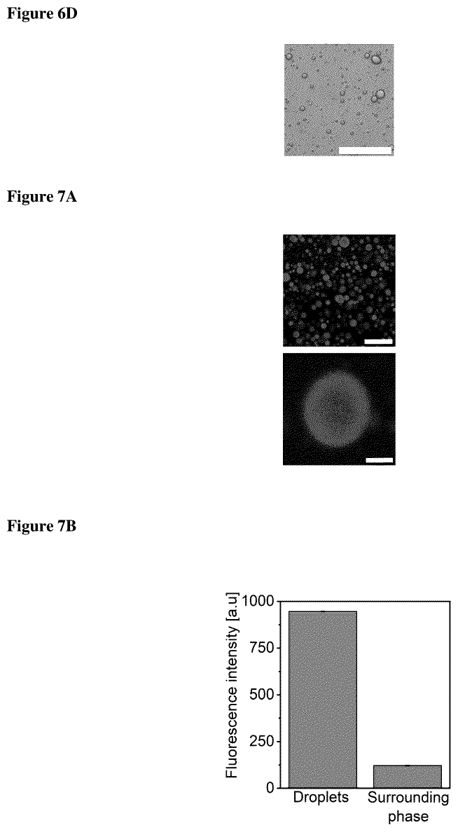

The liquid-liquid phase separation (LLPS) propensity of the WGR-6 peptide and its complexation with RNA to form liquid droplets was studied. Poly-uridylic acid (poly-U) was used as a model for RNA, (due to its low propensity to adopt a secondary structure), to promote complexation with the peptide. WGR-6 was dissolved in Tris buffer (pH 7.5) at varying concentrations between 0.2 mM to 5 mM and was mixed with poly-U at concentrations ranging from 0.025 mg/ml to 1 mg/ml. The turbidity of each peptide-RNA mixture was measured (λ=600 nm), and droplets formation was confirmed using optical microscopy. FIG. 5 shows the phase diagram of WGR-6-poly-U as a heat map of a colored optical density (OD) scale. The driving force which underlies droplets formation is electrostatic interaction between the positively charged peptide (basic arginine side chain) and the negatively charged phosphate groups of the poly-U. The turbidity of the mixtures indicated droplets formation as was verified by optical microscopy analysis (FIG. 6) which shows droplets with a diameter of 5.04±1.64 μm.

The enzyme tyrosinase (extracted from Agaricus bisporus) was used to oxidize the tyrosine side chains within the peptide assemblies. Oxidation of the tyrosine phenol side chain by tyrosinase should result in formation of catechol and quinone group side chains which, in turn, lead to polymerization of the peptide. This was expected to result in significant changes to the dynamic structure at the molecular level and in emerging optical properties.

The partitioning of tyrosinase in the peptide droplets was studied by confocal microscopy analysis. The enzyme is negatively charged at neutral pH (isoelectric point=4.7-5), therefore is expected to interact with the basic peptide. To monitor the partitioning of tyrosinase, the enzyme was labelled with Atto 633. FIG. 7a-b shows that the enzyme partitions>5-fold more in the peptide: RNA droplets compared to the surrounding phase.

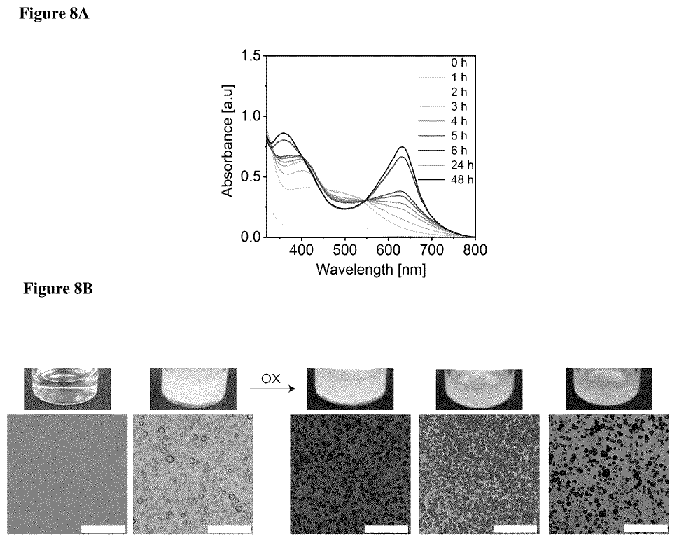

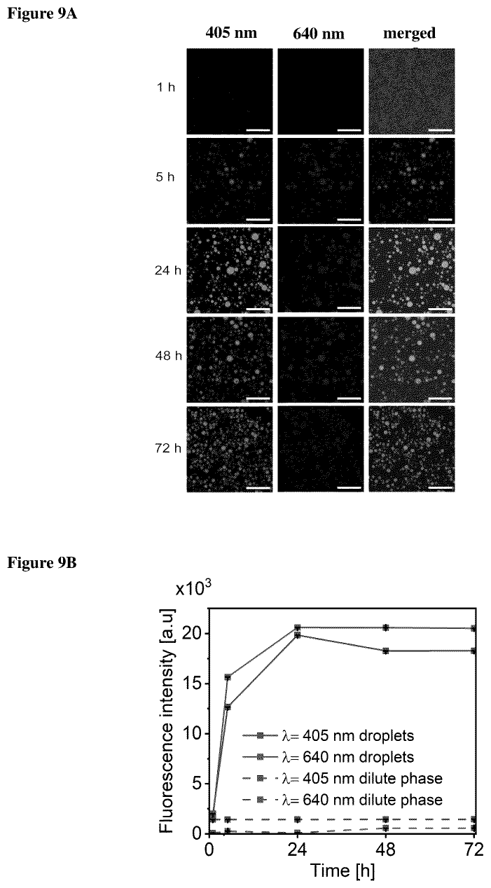

Next, UV-Vis spectroscopy measurements were done to monitor the changes in absorption upon oxidation-polymerization of the peptide droplets. The UV-Vis absorbance of peptide droplets formed by 2 mM WGR-6 and 1 mg/ml poly-U was monitored. Tyrosinase was added to pre-formed droplets at a final concentration of 0.1 mg/ml, and the UV-Vis spectra of the mixtures was taken over time (FIG. 8a). A significant broadening and increased intensity of the absorbance at λ=500 nm was observed after 1 h of oxidation, suggesting that the oxidized peptide further polymerized at this time. 2 h after the addition of tyrosinase, additional broad absorbance at λ=400 nm and λ=620 nm was observed. The intensity of these maxima increases over oxidation time between 2 h to 24 h, where at t=24 h, clear and distinctive maxima were observed, indicating on two dominant populations of polymeric assemblies at this time oxidation time. Macroscopic images of the material over oxidation time (FIG. 8b) show a color change of the reaction mixture to light pink immediately upon addition of tyrosinase. This color changes to light brown after 6 h of oxidation, corresponding to the broadening and increase in UV-Vis absorbance intensity. After 24 h of oxidation, a distinctive green color is observed, which corresponds to the absorbance at λ=620 nm.

Based on the distinctive absorption of the oxidized peptide droplets, the fluorescence of the droplets following oxidation using confocal microscopy was analyzed (FIG. 5). The droplets formed by 2 mM WGR-6 and 1 mg/ml poly-U with 0.1 mg/ml of unlabeled tyrosinase. The fluorescence of the droplets and surrounded phase over time: after 1 h, 4 h and 24 h of oxidation at λₑₓ=405 nm and λₑₓ=640 nm. A significant increase in the droplets' fluorescence intensity was observed over time as a result of the oxidation-polymerization by tyrosinase, where the fluorescence at λₑₓ=405 nm showed higher intensity.

To summarize, there is provided a method to form dynamic melanin-like assemblies by using a tyrosine-containing peptide, which forms liquid droplets by LLPS with RNA. The droplets sequester the enzyme tyrosinase, which in turn oxidizes the tyrosine side chain within the peptide building block. This enzymatic oxidation of the droplets results in emerging optical properties including coloration, UV-Vis absorbance, and fluorescence in the far-red while retaining microdroplet morphology. The reported technology offers a new strategy for synthesis of melanin-like assemblies, based on reaction compartmentalization and mimicry of melanin biosynthesis in cellular organelles.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. It is to be understood that further trials are being conducted to establish clinical effects.

1. A peptide liquid droplet, wherein the peptide liquid droplet comprises: a sequence comprising one or more dyad of arginine-glycine (RG) and one or more aromatic amino acid.

2. The peptide liquid droplet of claim 1, wherein the one or more aromatic amino acid is located at each end of the one or more dyad of arginine-glycine (RG).

3. The peptide liquid droplet of claim 1, wherein the aromatic amino acid is tyrosine, histidine, tryptophan or phenylalanine.

4. The peptide liquid droplet of claim 1, further comprising an ELP domain.

5. The peptide liquid droplet of claim 1, wherein valine at the first position of the ELP domain is substituted by tryptophan so that WPGVG is formed.

6. The peptide liquid droplet of claim 1, wherein the peptide liquid droplet comprises about 2-10 dyads of RG.

7. The peptide liquid droplet of claim 1, wherein the peptide liquid droplet comprises a sequence comprising
(SEQ ID No. 1)
WGRGRGRGWPGVGY;
(SEQ ID No. 2)
WGRGRGRGWPGVGF;
(SEQ ID No. [[3]]4)
WGRGRGRGWY;
(SEQ ID No. [[4]]5)
WGRGRGWPGVGY;
or
(SEQ ID No. 25)
WGRGRGRGWQGVGY;
(SEQ ID No. 6)
WGRGRGRWY.

8. The peptide liquid droplet of claim 1, wherein the peptide comprises between 5-50 amino acid

9. The peptide liquid droplet of claim 1, wherein the peptide is selected from sequences as set forth in Table 1.

10. The peptide liquid droplet of claim 1, wherein the peptide liquid droplet encapsulates an active agent.

11. A composition comprising a phase comprising the peptide liquid droplet of claim 1, and a dilute aqueous phase.

12. (canceled)

13. The peptide liquid droplet of claim 10, wherein the active agent is selected from the group consisting of proteins, (poly)peptides, carbohydrates, nucleic acids, lipids, chemical compounds, nanoparticles, and combinations thereof.

14. The peptide liquid droplet of claim 10, wherein the active agent is a pharmaceutical or diagnostic agent.

15. The composition of claim 11, wherein the pH of the composition is about 5-10.

16. (canceled)

17. (canceled)

18. The composition of claim 11, wherein the concentration of the peptide liquid droplet in the solution ranges between about 5 mM to about 30 mM.

19. (canceled)

20. (canceled)

21. A method for treating disease in a subject in need thereof, comprising: (i) administering a composition comprising the peptide liquid droplet according to claim 11 to the subject; and (ii) exposing the peptide liquid droplet to conditions that trigger the release of the pharmaceutical or diagnostic agent.

22. The method of claim 21, wherein the conditions that trigger the release of the pharmaceutical or diagnostic agent are selected from the group consisting of elevated temperatures, pH changes, exposure to release agents, exposure to gest molecule, exposure to light, enzymatic activation, and combinations thereof.

23. The peptide liquid droplet of claim 4, wherein the ELP domain is mixed together with a negatively charged polymer.

24. (canceled)

25. (canceled)

26. The peptide droplet of claim 23, wherein the negatively charged polymer is an RNA or DNA polymer.

27. The peptide droplet of claim 26, wherein the RNA polymer is a poly U polymer.

28.-31. (canceled)


SEQUENCE LISTING (28 sequences)

SEQ ID NO: 1 - protein, 14 aa, synthetic construct
WGRGRGRGWPGVGY

SEQ ID NO: 2 - protein, 14 aa, synthetic construct
WGRGRGRGWPGVGF

SEQ ID NO: 3 - protein, 14 aa, synthetic construct
WGRGRGRGWPGVGF

SEQ ID NO: 4 - protein, 10 aa, synthetic construct
WGRGRGRGWY

SEQ ID NO: 5 - protein, 12 aa, synthetic construct
WGRGRGWPGVGY

SEQ ID NO: 6 - protein, 9 aa, synthetic construct
WGRGRGRWY

SEQ ID NO: 7 - protein, 9 aa, synthetic construct
WGRGRGRGY

SEQ ID NO: 8 - protein, 8 aa, synthetic construct
WGRGRGWY

SEQ ID NO: 9 - protein, 14 aa, synthetic construct
WGRGRGRGWPGVGW

SEQ ID NO: 10 - protein, 14 aa, synthetic construct
WGRGRGRGYPGVGY

SEQ ID NO: 11 - protein, 14 aa, synthetic construct
YGRGRGRGYPGVGY

SEQ ID NO: 12 - protein, 14 aa, synthetic construct
WGRGRGRGFPGVGY

SEQ ID NO: 13 - protein, 14 aa, synthetic construct
WGRGRGRGFPGVGF

SEQ ID NO: 14 - protein, 14 aa, synthetic construct
FGRGRGRGFPGVGF

SEQ ID NO: 15 - protein, 10 aa, synthetic construct
WGRGRGRGWF

SEQ ID NO: 16 - protein, 10 aa, synthetic construct
FGRGRGRGWY

SEQ ID NO: 17 - protein, 10 aa, synthetic construct
WGRGRGRGYY

SEQ ID NO: 18 - protein, 10 aa, synthetic construct
FGRGRGRGFY

SEQ ID NO: 19 - protein, 9 aa, synthetic construct
WRGRGRGWY

SEQ ID NO: 20 - protein, 7 aa, synthetic construct
WRGRGWY

SEQ ID NO: 21 - protein, 7 aa, synthetic construct
WRGRGWW

SEQ ID NO: 22 - protein, 7 aa, synthetic construct
WRGRGFY

SEQ ID NO: 23 - protein, 7 aa, synthetic construct
FRGRGFY

SEQ ID NO: 24 - protein, 14 aa, synthetic construct
WGKGKGKGWPGVGY

SEQ ID NO: 25 - protein, 14 aa, synthetic construct
WGRGRGRGWQGVGY

SEQ ID NO: 26 - protein, 6 aa, synthetic construct
RGRGRG

SEQ ID NO: 27 - protein, 5 aa, synthetic construct
WPGVG

SEQ ID NO: 28 - protein, 5 aa, synthetic construct
WQGVG